(19) United States
(12) Patent Application Publication
(10) Pub. No.: US 2026/0041319 A1
(43) Pub. Date: Feb. 12, 2026

(54) METHOD AND OPTICAL SYSTEM FOR DETERMINING A RISK OF THE PRESENCE OF AN EYE DISEASE

(71) Applicant: Robert Bosch GmbH, Stuttgart (DE)

(72) Inventors: Carsten Reichert, Gomaringen (DE); Thomas Alexander Schlebusch, Renningen (DE)

(21) Appl. No.: 19/273,544

(22) Filed: Jul. 18, 2025

(30) Foreign Application Priority Data
Aug. 6, 2024 (DE) 10 2024 207 415.2

(51) Int. Cl. A61B 3/12; A61B 3/00

(52) U.S. Cl. CPC A61B 3/12; A61B 3/0008; A61B 3/0025

(57) ABSTRACT

A method for determining a risk of the presence of an eye disease. At a first point in time, a first infrared light beam is emitted using a laser feedback interferometer sensor. The first infrared light beam is scanned over a first region of a retina of an eye using at least one micromirror. The first infrared light beam reflected back from the retina is detected using the laser feedback interferometer sensor. First polarization signals of the first region are ascertained according to a self-mixing effect. At a second point in time following the first point in time, the method steps are repeated and a second infrared light beam is emitted. The risk of the presence of the eye disease is determined according to a comparison of the ascertained first polarization signals with the ascertained second polarization signals.

### CROSS REFERENCE

The present application claims the benefit under 35 U.S.C. § 119 of Germany Patent Application No. DE 10 2024 207 415.2 filed on Aug. 6, 2024, which is expressly incorporated herein by reference in its entirety.

### FIELD

The present invention relates to a method for determining a risk of the presence of an eye disease. Furthermore, the present invention relates to an optical system for determining a risk of the presence of an eye disease.

### BACKGROUND INFORMATION

European Patent No. EP 2 702 934 B1 describes the relationship between decreasing retinal thickness and the birefringence generated by the retina.

It is an object of the present invention to develop a simple and error-free method for determining a risk of the presence of an eye disease.

### SUMMARY

To achieve the object, a method for determining a risk of the presence of an eye disease is provided. Furthermore, an optical system for determining a risk of the presence of an eye disease is provided.

According to an example embodiment of the present invention, in the method for determining a risk of the presence of an eye disease, initially, at least one first infrared light beam is emitted by means of a laser feedback interferometer sensor at a first point in time. The laser feedback interferometer sensor is a VCSEL with an integrated photodiode. In a further method step, the at least one first infrared light beam is scanned over a first region of a retina of an eye by means of at least one micromirror that is mounted so as to be rotatable in at least one dimension.

The micromirror is in particular a micromirror mounted so as to be rotatable in two dimensions or two micromirrors each mounted so as to be rotatable in one dimension. Furthermore, the first infrared light beam reflected back from the retina is detected by means of the laser feedback interferometer sensor. In a further method step, first polarization signals of the first region of the retina of the eye are ascertained by means of a computing unit according to a self-mixing effect. This self-mixing effect is described in particular as an interference of the detected back-reflected first infrared light beam with a light wave located in a laser cavity of the laser feedback interferometer sensor. In a further method step, at least one second infrared light beam is emitted by means of the laser feedback interferometer sensor at a second point in time following the first point in time. Furthermore, the at least one second infrared light beam is scanned over the first region of the retina of the eye by means of the at least one micromirror. In a further method step, the second infrared light beam reflected back from the retina is detected by means of the laser feedback interferometer sensor. Furthermore, second polarization signals of the first region of the retina of the eye are ascertained by the computing unit by means of the self-mixing effect. Here as well, the self-mixing effect is in particular an interference of the detected back-reflected second infrared light beam with a light wave located in a laser cavity of the laser feedback interferometer sensor. In a further method step, the risk of the presence of the eye disease is determined by means of the computing unit according to a comparison of the ascertained first polarization signals of the first region of the retina of the eye with the ascertained second polarization signals of the first region of the retina of the eye. Since the eye, in particular the retina, generates a polarization change of the laser signal of the LFI laser signal, a change in the degree of polarization change over time causes a change in the signal strength of the measurement data. If the retina changes due to an eye disease, e.g. becomes thinner, then this has an influence on the polarization change generated by the retina, and so the change in the degree of polarization change can be an indication of the risk of the eye disease. Preferably, before the first infrared light beam is emitted for the first time, a suitable normalization of the signal strength takes place, in particular by means of information about a position of the pupil in the coordinate system of a pair of smart glasses and/or by means of information about the eye, in particular a size of the pupil.

Preferably, the eye disease is glaucoma, in particular green star, of the eye. In this connection, according to an example embodiment of the present invention, the method preferably additionally ascertains a change in a layer thickness, in particular a decrease, of the first region of the retina according to the comparison of the ascertained first polarization signals of the first region of the retina of the eye with the ascertained second polarization signals of the first region of the retina of the eye by means of the computing unit. Furthermore, the risk of the presence of glaucoma is determined according to a comparison of the ascertained change in layer thickness with a threshold value by means of the computing unit. If a decrease in the thickness of the retina above the threshold value is established, in particular within a defined period of time, this may indicate the death of nerve fibers, which in turn is an indication of glaucoma.

According to an example embodiment of the present invention, preferably, the first and second points in time are at least two weeks and at most one year apart. Within this time frame, meaningful results about the risk of the presence of the eye disease can be obtained.

According to an example embodiment of the present invention, preferably, the first region of the retina is arranged in the region of the optic nerve and comprises the optic nerve. The retina is very thick in the region of the optic nerve, which makes it particularly easy to ascertain changes in the thickness of the retina there. Furthermore, the optic nerve can also be used as a kind of orientation point for the location of the first region within the entire retina of the eye.

According to an example embodiment of the present invention, preferably, furthermore, a first, in particular visible, geometric shape is projected onto a first, in particular central, position of a field of view of a user of a pair of smart glasses at a third point in time prior to the first point in time by means of a light unit and the at least one micromirror. The projection is therefore the so-called retinal projection and the first geometric shape is in particular designed as a first rectangle. In a further method step, the first infrared light beam is emitted at the first point in time when the user of the smart glasses looks at the first geometric shape. In particular, the first infrared light beam is emitted when the first geometric shape is in the center of the field of view of the user of the smart glasses. Thus, first polarization signals can be ascertained from a certain specified first region of the retina as a reference for future measurements in this region. In a further method step, a second, in particular visible, geometric shape is projected onto a second, in particular lateral, position of the field of view of the user of the smart glasses at a fourth point in time, which is after the first point in time and prior to the second point in time, by means of the light unit and the at least one micromirror. The projection here is therefore also the so-called retinal projection and the second geometric shape is in particular designed as a second rectangle. Furthermore, a third infrared light beam is emitted by means of the laser feedback interferometer sensor at a fifth point in time, which is after the fourth point in time and prior to the second point in time, when the user of the smart glasses looks at the second geometric shape. In particular, the second infrared light beam is emitted when the second geometric shape is in the center of the field of view of the user of the smart glasses. Thus, third polarization signals can be ascertained from a certain specified second region of the retina as a reference for future measurements on this region. The method steps are then repeated, in particular at the second point in time, in order to perform a comparison measurement with the previously determined reference values in the predetermined ranges.

According to an example embodiment of the present invention, as an alternative to the previous possibility, an optic nerve of the eye is preferably detected by means of the laser feedback interferometer sensor in the first region of the retina at the first point in time. Furthermore, the viewing direction of the eye at the first point in time is ascertained by means of the laser feedback interferometer sensor. In a further method step, the first region of the retina of the eye is ascertained by means of the computing unit according to a position of the detected optic nerve and the ascertained viewing direction.

These method steps make it possible to determine the risk of eye disease automatically in the background. The eye does not have to carry out any specified eye movements for this purpose; rather, at the beginning, only the optic nerve in the detected image and the current viewing direction of the eye are required for orientation. Furthermore, the laser feedback interferometer sensor can be used for ascertaining the polarization signals and for ascertaining the viewing direction. The method is thus simple and requires only a few components to execute.

Preferably, the risk of the presence of the eye disease is determined if the identified first region at the second point in time corresponds at least partially to the first region identified at the first point in time. To ensure comparability of the results, at least partially identical regions of the retina are required at the first and second points in time. This is the only way to really say whether there has been a change in the thickness of the retina. Preferably, further first regions of the retina of the eye are identified at further points in time following the first point in time according to a change in the viewing direction of the eye detected by means of the laser feedback interferometer sensor. The optic nerve is only required in the detected image for initial orientation. Subsequently, by detecting the change in the viewing direction, the first region can be identified even without the optic nerve in the image.

According to an example embodiment of the present invention, preferably, the risk of the presence of the eye disease is determined when a user of a pair of smart glasses puts on the smart glasses. The smart glasses can shift over time when worn on the nose of the smart glasses user, making a comparison with previously measured reference values difficult. Determining the risk of the presence of the eye disease each time the user puts on the smart glasses has the advantage that the glasses are in substantially the same position relative to the user's head at such point in time. A comparison of the measurement results thus makes sense at such point in time.

According to an example embodiment of the present invention, preferably, the determined risk of the presence of the eye disease in a person associated with the eye is displayed via a display unit, in particular visually or acoustically. In particular, the display unit is a pair of smart glasses.

Furthermore, the display unit is a wearable item, such as a smart watch. The display unit can also be a smart phone. In particular, recommendations for action can be displayed to the person. Such a recommendation for action could, for example, be as follows: “You have an increased risk of having an eye disease, in particular glaucoma. Please go to an eye doctor and have this checked.”

According to an example embodiment of the present invention, a further object of the present invention is an optical system for determining a risk of the presence of an eye disease. Here, the optical system comprises a laser feedback interferometer sensor, a computing unit and at least one micromirror mounted so as to be rotatable in at least one dimension. The laser feedback interferometer sensor is designed to emit at least one first infrared light beam at a first point in time. The micromirror serves to scan the at least one first infrared light beam over a first region of a retina of an eye. The laser feedback interferometer sensor is designed to detect the first infrared light beam reflected back from the retina. The computing unit serves to ascertain first polarization signals of the first region of the retina of the eye according to a self-mixing effect. The self-mixing effect is in particular an interference of the detected back-reflected first infrared light beam with a light wave located in a laser cavity of the laser feedback interferometer sensor. The laser feedback interferometer sensor is further designed to emit at least one second infrared light beam at a second point in time following the first point in time. The micromirror, in turn, serves to scan the at least one second infrared light beam over the first region of the retina of the eye. The laser feedback interferometer sensor is designed to detect the second infrared light beam reflected back from the retina. The computing unit, in turn, serves to ascertain second polarization signals of the first region of the retina of the eye according to the self-mixing effect. In this connection, the self-mixing effect is in particular an interference of the detected back-reflected second infrared light beam with a light wave located in a laser cavity of the laser feedback interferometer sensor. Furthermore, the computing unit is designed to determine the risk of the presence of the eye disease according to a comparison of the ascertained first polarization signals of the first region of the retina of the eye with the ascertained second polarization signals of the first region of the retina of the eye.

According to an example embodiment of the present invention, preferably, the laser feedback interferometer sensor is additionally designed to ascertain a viewing direction of the eye. Thus, the laser feedback interferometer sensor can carry out a plurality functions.

According to an example embodiment of the present invention, preferably, the optical system is designed as a pair of smart glasses. In this connection, the optical system additionally comprises a light unit, in particular a laser diode, for emitting visible light beams. The light unit and the micromirror serve to project a first, in particular visible, geometric shape, in particular a first rectangle, onto a first, in particular central, position and a second, in particular visible, geometric shape, in particular a second rectangle, onto a second, in particular lateral, position of a field of view of the user of the smart glasses. The light unit and the micromirror are therefore designed as a projection unit, in particular as a microscanner unit. In order to deflect the visible light beams onto the retina of the eye of the smart glasses user, a deflection unit is further provided in particular, which is designed as a holographic optical element.

### BRIEF DESCRIPTION OF THE DRAWINGS

FIG. 1 shows a first method for determining a risk of the presence of an eye disease, according to an example embodiment of the present invention.

FIG. 2 shows an optical system for determining a risk of the presence of an eye disease, according to an example embodiment of the present invention.

FIG. 3 shows a first possibility for determining a risk of the presence of an eye disease, according to an example embodiment of the present invention.

FIGS. 4A and 4B show a second possibility for determining a risk of the presence of an eye disease, according to an example embodiment of the present invention.

FIGS. 5A and 5B show a second automatic possibility for determining a risk of the presence of an eye disease, according to an example embodiment of the present invention.

### DETAILED DESCRIPTION OF EXAMPLE EMBODIMENTS

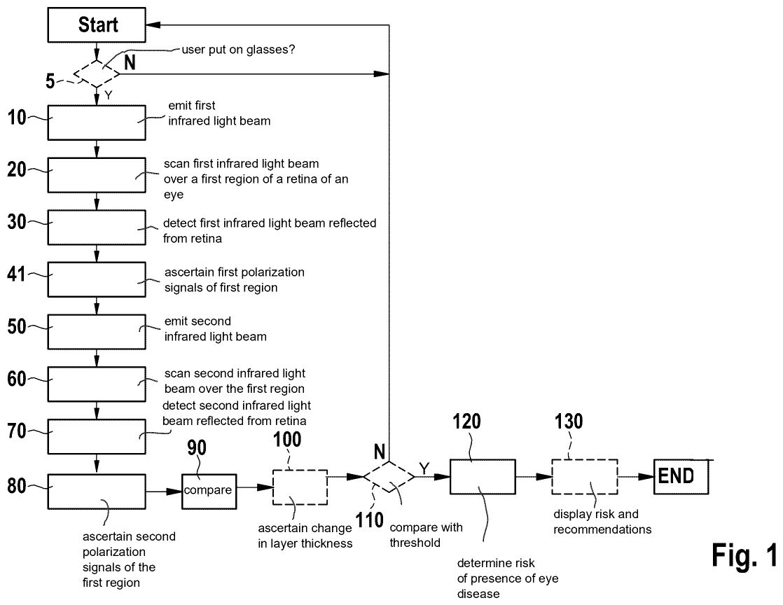

FIG. 1 shows a method for determining a risk of the presence of an eye disease in the form of a flow chart. In a method step 10, at least one first infrared light beam is emitted at a first point in time by means of a laser feedback interferometer sensor. In a subsequent method step 20, the at least one first infrared light beam is scanned over a first region of a retina of an eye by means of at least one micromirror mounted so as to be rotatable in at least one dimension. In a subsequent method step 30, the first infrared light beam reflected back from the retina is detected by means of the laser feedback interferometer sensor. In a further method step 41, first polarization signals of the first region of the retina of the eye are ascertained by means of a computing unit according to a self-mixing effect. In this connection, the self-mixing effect is in particular an interference of the detected back-reflected first infrared light beam with a light wave located in a laser cavity of the laser feedback interferometer sensor. In a further method step 50, at a second point in time following the first point in time, at least one second infrared light beam is emitted by means of the laser feedback interferometer sensor. In a subsequent method step 60, the at least one second infrared light beam is scanned over the first region of the retina of the eye by means of the at least one micromirror. In a further method step 70, the second infrared light beam reflected back from the retina is detected by means of the laser feedback interferometer sensor. In a subsequent method step 80, second polarization signals of the first region of the retina of the eye are ascertained by means of the computing unit according to the self-mixing effect. In this connection, the self-mixing effect is an interference of the detected back-reflected second infrared light beam with a light wave located in a laser cavity of the laser feedback interferometer sensor. In a subsequent method step 120, the risk of the presence of the eye disease is determined by means of the computing unit according to a comparison 90 of the ascertained first polarization signals of the first region of the retina of the eye with the ascertained second polarization signals of the first region of the retina of the eye. The method is then terminated.

Optionally, the eye disease is glaucoma, in particular green star, of the eye. In a further optional method step 100 following method step 90, a change in a layer thickness, in particular a decrease in the layer thickness, of the first region of the retina is ascertained by means of the computing unit according to the comparison in method step 90 of the ascertained first polarization signals of the first region of the retina of the eye with the ascertained second polarization signals of the first region of the retina of the eye. In an optional method step 110 following method step 100, the ascertained change in layer thickness is compared with a threshold value by means of the computing unit. Here, if it is established that the ascertained change, in particular the ascertained decrease, in the layer thickness is below the threshold value, the method is started again at a later point in time. However, if it is established here that the ascertained change, in particular the ascertained decrease, in the layer thickness is above the threshold value, there is an increased risk of the presence of glaucoma in the corresponding eye.

Optionally, the method is repeated at intervals of at least two weeks and a maximum of one year.

In an optional method step 130 following method step 120, the determined risk of the presence of the eye disease, and in particular recommendations for action, are displayed to a person associated with the eye by means of a display unit.

Optionally, the first region of the retina is arranged in the region of the optic nerve and comprises the optic nerve.

In a further optional method step 5, it is checked whether the user of a pair of smart glasses has just put the glasses on. Here, if it is determined that the glasses have been on the user's nose for a long time, the method is started again from the beginning. Here, the risk that the glasses have shifted on the nose, potentially leading to erroneous measurement comparisons, is too high. However, if it is established that the user has just put on the glasses, the method continues and the risk of the presence of the eye disease is determined.

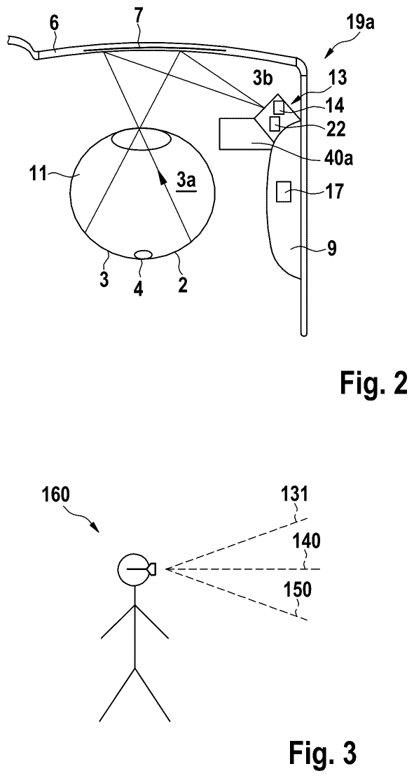

FIG. 2 schematically shows, in the form of a pair of smart glasses, an optical system 19a for determining a risk of the presence of an eye disease. Here, the optical system comprises a laser feedback interferometer sensor 40a, a computing unit 17 and a micromirror 14 mounted so as to be rotatable in two dimensions. In this embodiment, the computing unit 17 is integrated into a spectacle frame 9. The laser feedback interferometer sensor 40a is designed to emit at least one first infrared light beam 3b at a first point in time. The micromirror 14 serves to scan the at least one first infrared light beam 3b over a first region 2 of a retina 3 of an eye 11. The laser feedback interferometer sensor 40a is in turn designed to detect the first infrared light beam 3a reflected back from the retina 3. The computing unit 17 is designed to ascertain first polarization signals of the first region 2 of the retina 3 of the eye 11 according to a self-mixing effect, in particular an interference of the detected back-reflected first infrared light beam 3a with a light wave located in a laser cavity of the laser feedback interferometer sensor 40a. The laser feedback interferometer sensor 40a further serves to emit at least one second infrared light beam (not shown here for the sake of simplicity) at a second point in time following the first point in time. In this connection, the micromirror 14 is designed to scan the at least one second infrared light beam over the first region 2 of the retina 3 of the eye 11. The laser feedback interferometer sensor 40a serves to detect the second infrared light beam (not shown here) reflected back from the retina 3.

The computing unit 17 is designed to ascertain second polarization signals of the first region 2 of the retina 3 of the eye 11 according to the self-mixing effect, in particular an interference of the detected back-reflected second infrared light beam with a light wave located in a laser cavity of the laser feedback interferometer sensor 40a. Furthermore, the computing unit 17 serves to determine the risk of the presence of the eye disease according to a comparison of the ascertained first polarization signals of the first region 2 of the retina 3 of the eye 11 with the ascertained second polarization signals of the first region 2 of the retina 3 of the eye 11.

In this exemplary embodiment, the micromirror 14 and the laser feedback interferometer sensor 40a are integrated into a projector unit 13 of the optical system 19a. The projector unit 13 further comprises a light unit 22 for emitting visible light beams (not shown here), and is integrated into the spectacle frame 9. The light unit 22 and the micromirror 14 serve to project a first, in particular visible, geometric shape, in particular a first rectangle, onto a first, in particular central, position and a second, in particular visible, geometric shape, in particular a second rectangle, onto a second, in particular lateral, position of a field of view of the user of the smart glasses. In order to deflect the light beams onto the eye 11, the optical system additionally comprises a deflection unit 7. Here, the deflection unit 7 is designed as a holographic optical element which is integrated into a spectacle lens 6.

The laser feedback interferometer sensor 40a is optionally additionally designed to ascertain a viewing direction of the eye 11.

FIG. 3 schematically shows a first possibility for determining a risk of the presence of an eye disease. Here, at a third point in time prior to the first point in time, a first, visible geometric shape (not shown here) is projected onto a first, central position 140 of a field of view of the smart glasses user 160 by means of the light unit of FIG. 2 and the at least one micromirror of FIG. 2. The first geometric shape is, in particular, a first rectangle. The first infrared light beam is then emitted at the first point in time when it is determined that the smart glasses user 160 is actually looking at the projected geometric shape.

Furthermore, at a fourth point in time, which is after the first point in time and prior to the second point in time, a second, visible geometric shape is projected onto a second, upper position 131 of the field of view of the user of the smart glasses. The second geometric shape is in particular a second rectangle. A third infrared light beam is then emitted at a fifth point in time, after the fourth point in time and prior to the second point in time, by means of the laser feedback interferometer sensor if it is established that the smart glasses user is actually looking at the second rectangle. At a further point in time, a third, visible geometric shape is projected onto a third, lower position 150 of the field of view of the smart glasses. Thus, reference measurements of polarization signals of the respective retinal regions are recorded, which can then be compared with newly recorded measurements at a subsequent second point in time.

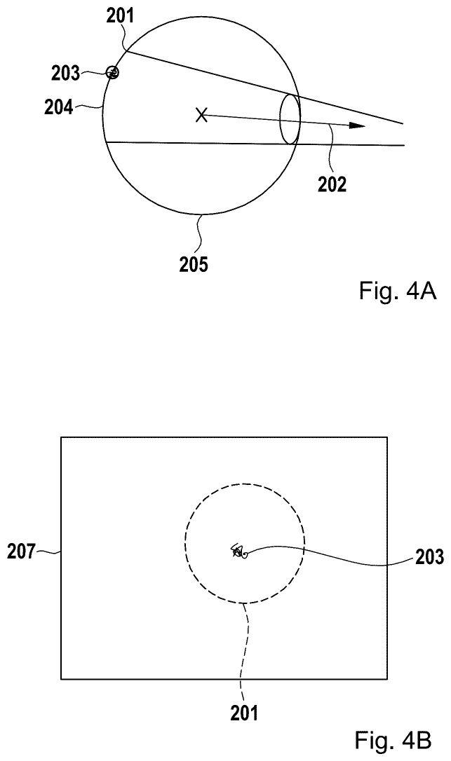

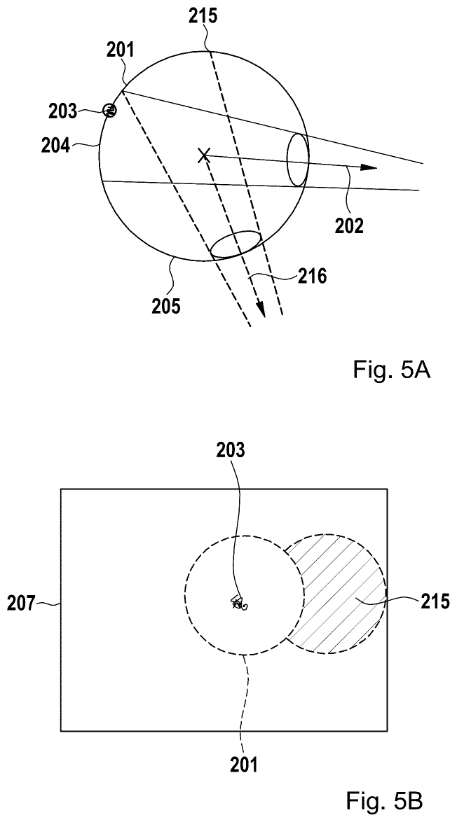

FIG. 4A schematically shows a second possibility for determining the risk of the presence of an eye disease. Here, at the first point in time, an optic nerve 203 of the eye 205 is detected in the first region 201 of the retina 204 by means of the laser feedback interferometer sensor (not shown here). Furthermore, at the first point in time, a viewing direction of the eye is ascertained in the form of a gaze vector 202 by means of the laser feedback interferometer sensor. According to a position of the detected optic nerve, in particular within the first region 201, and the ascertained viewing direction, the first region 201 of the retina 204 of the eye 205 is now identified. In particular, as shown in FIG. 4B, the first region 204 identified at the first point in time can be entered on a type of two-dimensional map 207 of the retina 204. As shown in FIG. 5A, at a further point in time following the first point in time, a further first region 215 of the retina of the eye can be identified according to a change in the viewing direction of the eye 205 detected by means of the laser feedback interferometer sensor. From FIG. 4A to FIG. 5A, the gaze vector 202 has changed to the new gaze vector 216. This information is sufficient to identify the further first region 215 and to plot it on the two-dimensional map 207 of the retina 204 in FIG. 5B. The optic nerve is no longer required for identification. In particular, the risk of the presence of the eye disease is only determined if the first regions 201 and 215 identified at different times at least partially coincide. In the example shown in FIG. 5B, there is no such correspondence or overlap between the first regions 201 and 215. Thus, the first identified regions 201 and 215 can only serve as reference regions for future measurements.

What is claimed is:

1. A method for determining a risk of a presence of an eye disease, the method comprising the following method steps:
emitting, at a first point in time, at least one first infrared light beam using a laser feedback interferometer sensor;
scanning the at least one first infrared light beam over a first region of a retina of an eye using at least one micromirror mounted so as to be rotatable in at least one dimension;
detecting the first infrared light beam reflected back from the retina using the laser feedback interferometer sensor;
ascertaining, using a computing unit, first polarization signals of the first region of the retina of the eye according to a self-mixing effect, including an interference of the detected back-reflected first infrared light beam with a light wave located in a laser cavity of the laser feedback interferometer sensor;
emitting, at a second point in time following the first point in time, at least one second infrared light beam using the laser feedback interferometer sensor;
scanning the at least one second infrared light beam over the first region of the retina of the eye using the at least one micromirror;
detecting the second infrared light beam reflected back from the retina using the laser feedback interferometer sensor;
ascertaining, using the computing unit, second polarization signals of the first region of the retina of the eye according to the self-mixing effect, including an interference of the detected back-reflected second infrared light beam with a light wave located in a laser cavity of the laser feedback interferometer sensor; and
determining, using the computing unit, the risk of the presence of the eye disease according to a comparison of the ascertained first polarization signals of the first region of the retina of the eye with the ascertained second polarization signals of the first region of the retina of the eye.

2. The method according to claim 1, characterized in that the eye disease is glaucoma of the eye.

3. The method according to claim 2, further comprising the following steps:
ascertaining, using the computing unit, a change in a layer thickness, including a decrease, of the first region of the retina according to the comparison of the ascertained first polarization signals of the first region of the retina of the eye with the ascertained second polarization signals of the first region of the retina of the eye; and
determining, using the computing unit, the risk of the presence of glaucoma according to a comparison of the ascertained change in the layer thickness with a threshold value.

4. The method according to claim 1. Wherein the first and second points in time are at least two weeks and at most one year apart.

5. The method according to claim 1, wherein the first region of the retina is arranged in a region of an optic nerve and includes the optic nerve.

6. The method according to claim 1, wherein the method further comprises the following additional steps:
projecting a first visible geometric shape, including a first rectangle, onto a first central position of a field of view of a user of a pair of smart glasses at a third point in time prior to the first point using a light unit and the at least one micromirror;
emitting the first infrared light beam at the first point in time when the user of the smart glasses looks at the first geometric shape;
projecting a second visible geometric shape, including a second rectangle, onto a second lateral position of the field of view of the user of the smart glasses at a fourth point in time, which is after the first point in time and prior to the second point in time, using the light unit and the at least one micromirror; and
emitting a third infrared light beam using the laser feedback interferometer sensor at a fifth point in time, which is after the fourth point in time and prior to the second point in time, when the user of the smart glasses looks at the second geometric shape; and
repeating the additional method steps at the second point in time.

7. The method according to claim 1, wherein the method further comprises the following additional method steps:
detecting an optic nerve of the eye using the laser feedback interferometer sensor in the first region of the retina at the first point in time;
ascertaining a viewing direction of the eye at the first point in time using the laser feedback interferometer sensor; and
identifying, using the computing unit, the first region of the retina of the eye according to a position of the detected optic nerve and the ascertained viewing direction.

8. The method according to claim 7, wherein the risk of the presence of the eye disease is determined when the identified first region at the second point in time at least partially coincides with the first region identified at the first point in time.

9. The method according to claim 7, wherein at further points in time following the first point in time, further first regions of the retina of the eye are identified according to a change in viewing direction of the eye detected using the laser feedback interferometer sensor.

10. The method according to claim 1, wherein the risk of the presence of the eye disease is determined when a user of a pair of smart glasses puts on the smart glasses.

11. The method according to claim 1, wherein the determined risk of the presence of the eye disease, and recommendations for action, are displayed to a person associated with the eye using a display unit.

12. An optical system for determining a risk of the presence of an eye disease, comprising:
a laser feedback interferometer sensor;
a computing unit; and
at least one micro-mirror that is mounted so as to be rotatable in at least one dimension;
wherein the laser feedback interferometer sensor is configured to emit at least one first infrared light beam at a first point in time, wherein the micromirror is configured to scan the at least one first infrared light beam over a first region of a retina of an eye, wherein the laser feedback interferometer sensor is configured to detect the first infrared light beam reflected back from the retina, wherein the computing unit is configured to generate first polarization signals of the first region of the retina of the eye according to a self-mixing effect, including an interference of the detected back-reflected first infrared light beam with a light wave located in a laser cavity of the laser feedback interferometer sensor; wherein the laser feedback interferometer sensor is configured to emit at least one second infrared light beam at a second point in time following the first point in time, wherein the micromirror is configured to scan the at least one second infrared light beam over the first region of the retina of the eye, wherein the laser feedback interferometer sensor is configured to detect the second infrared light beam reflected back from the retina, wherein the computing unit is configured to generate second polarization signals of the first region of the retina of the eye according to the self-mixing effect, including an interference of the detected back-reflected second infrared light beam with a light wave located in a laser cavity of the laser feedback interferometer sensor, and to determine the risk of the presence of the eye disease according to a comparison of the ascertained first polarization signals of the first region of the retina of the eye with the ascertained second polarization signals of the first region of the retina of the eye.

13. The optical system according to claim 12, wherein the laser feedback interferometer sensor is additionally configured to ascertain a viewing direction of the eye.

14. The optical system according to claim 12, wherein the optical system is a pair of smart glasses.

15. The optical system according to claim 14, wherein the optical system additionally comprises a light unit configured to emit visible light beams, wherein the light unit and the micromirror serve to project a first visible geometric shape, including a first rectangle, onto a first central position and a second visible geometric shape, including a second rectangle, onto a second lateral, position of a field of view of the user of the smart glasses.
